(19) United States
(12) Patent Application Publication
(10) Pub. No.: US 2025/0025607 A1
(43) Pub. Date: Jan. 23, 2025

(54) METHOD FOR PREPARING DRUG ELUTING BALLOONS WITHOUT COATING

(71) Applicant: SINO MEDICAL SCIENCES TECHNOLOGY, INC., Tianjin (CN)

(72) Inventors: Christophe BUREAU, Tianjin (CN); Ran SONG, Tianjin (CN); Xiaomei ZHANG, Tianjin (CN); Zhongcheng PAN, Tianjin (CN); Hongtao FAN, Tianjin (CN)

(73) Assignee: SINO MEDICAL SCIENCES TECHNOLOGY, INC., Tianjin (CN)

(21) Appl. No.: 18/714,809

(22) PCT Filed: Feb. 23, 2023
(86) PCT No.: PCT/CN2023/077962

§ 371 Date: May 30, 2024

Related U.S. Application Data
(60) Provisional application No. 63/313,221, filed on Feb. 23, 2022.

(51) Int. Cl. A61L 29/16; A61L 29/06

(52) U.S. Cl. CPC A61L 29/16; A61L 29/06

(57) ABSTRACT

A drug eluting balloon loaded with a drug in bulk, without any extra coating to host the said drug, and the method of preparing a drug eluting balloon which directly loads the drug on the balloon without coating, by using supercritical carbon dioxide to dissolve the drug and impregnate the balloons. The carbon dioxide is then released by pressure reduction, leaving only the drug on the balloon. Under suitable processing conditions, this method can form the drug uniformly on the surface of the balloon while maintaining the exterior morphology of the balloon, with a minimal increase in balloon thickness, and achieve satisfactory drug loading.

### BACKGROUND OF THE INVENTION

### Field of the Invention

The present invention relates to the field of medical devices and specifically to a method of preparing a drug eluting balloon without coating or a method of loading a drug onto the surface of a balloon without coating.

### Description of Related Art

Drug eluting balloon is a therapeutic balloon drug release technique developed on the basis of interventional medicine such as balloon dilation or balloon angioplasty. The mechanism of action of this drug-loaded balloon is to coat the surface of the balloon uniformly with an anti-cell proliferation drug, transport it to the site of vascular lesion, inflate the balloon in the blood vessel during a short time (30-60s), so that the vessel diameter is increased and the drug is released and adheres and/or penetrates into the vessel wall, and then deflate the balloon and withdraw it from the artery. The drug meant to help to prevent the restenosis traditionally following this balloon angioplasty is the one unloaded from the balloon during its expansion at the lesion site. This unloading must hence happen rapidly over the 30-60 seconds that the balloon is deployed: some mechanism must take place so that the drug is not washed away by the blood flow after the balloon is removed, i.e. the drug must adhere to or be embedded in a material that itself adheres to the tissues, or penetrate the tissue rapidly enough so that it remains at the lesion site.

In order to be used even when the primary stenosis or narrowing of the artery is significant, the diameter of a cross-section of the folded balloon—also known as the “profile” of the balloon—must be as small as possible. The smaller the diameter of the profile, the better the ability of the balloon to pass through stenotic lesions and reopen vessels. Typically, this profile is lower than 1 millimeter in the folded state.

There are essentially two approaches to designing drug eluting balloon: via coatings, or via multi-wall balloons.

When coatings are used, an additional thickness is introduced—on the order of a few micrometers to a few tens of micrometers—associated with the layer that contains the drug to be released. Drug-coated balloons have to go through interventional procedures to enter the human vasculature, and they have to go through challenges such as blood flow flushing, catheters, guidewires, and friction of stenotic lesions before reaching the lesion site, and many drug eluting balloons have already lost serious amounts of their drugs by the time they reach the lesion site, thus failing to achieve significant therapeutic effects. In order to compensate for this loss of drug and to maintain sufficient drug release at the site of vascular lesion, the initial design of the drug coated balloons intentionally increases the drug load, which increases the thickness of the balloon even more, making it not only difficult to reach highly closed lesions, but also increasing the toxicity of the excess drug to the vascular cells.

In addition, due to folding of the balloon, this extra-thickness is superimposed at least 3 times in some zones, and the radius of curvature at the folds is also larger than without coating in order to avoid cracking of the coating: as a result, the profile of the folded coated balloon is usually larger than that of the bare balloon equivalent. In some designs, the drug loaded coating is over-coated with one or more sacrificial layers, on a non-folded or folded balloon, in order to control the erosion of the coating over winding paths and during expansion, and thus minimize drug loss before the lesion is reached. However, although the large loss of drug in reaching the lesion site is avoided, the thickness of the drug balloon coating is also increased, making it more difficult for the balloon to reach highly closed lesions due to the larger diameter that needs to be passed.

Additionally, common drug-coated balloons may have the drug coating damaged during the folding process, and sometimes the coating that has been sprayed is not strong, and the drug is partially lost after balloon folding. A large amount of organic solvent is also used while forming the coating.

When multi-walled balloons are used, the outer wall balloon contains a plurality of holes, and the drug to be released is squeezed between this outer balloon and an inner balloon: when the multi-walled balloon is inflated, the inner balloon presses the volume between the two balloons, and expels the drug compound through the holes onto the artery walls. Again, due to the multi-layer structure of the balloon, the global thickness of the multi-walled balloon is larger than that of a single-wall balloon, and upon folding, the resulting drug eluting balloon has a profile with a diameter larger than the bare balloons.

### SUMMARY

Thus, there is a need to provide a method of preparing drug eluting balloons that minimizes the resulting profile diameter of the balloon without compromising the quantity of drug loading.

Additional features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

To achieve the above objects, the present invention provides a method of preparing a drug eluting balloon, which includes: preparing a balloon; dissolving a drug or a compound or mixture containing the drug in a supercritical carbon dioxide; impregnating the balloon with the supercritical carbon dioxide having the drug dissolved therein in a reactor chamber; and removing the supercritical carbon dioxide from the reactor chamber by depressurization.

In some embodiments, the balloon is formed of a polyether block amide or polyN-vinylpyrrolidone.

In some embodiments, the balloon is formed of a polyether block amide, the supercritical carbon dioxide is maintained at a temperature between 35 and 55 T and a pressure between 80 and 250 bars during impregnation, and the impregnating step is maintained for a time duration of 30 minutes to 4 hours.

In some embodiments, the drug is selected from of sirolimus, sirolimus derivatives, sirolimus analogs, inhibitory RNA, inhibitory DNA, steroids, and complement inhibitors. In some embodiments, the drug is sirolimus.

It is to be understood that both the foregoing general description and the following detailed description are exemplary and explanatory and are intended to provide further explanation of the invention as claimed.

### BRIEF DESCRIPTION OF THE DRAWINGS

FIG. 1 schematically illustrates a system for preparing drug eluting balloons using supercritical carbon dioxide impregnation according to an embodiment of the present invention.

FIG. 2 is a flow chart that schematically illustrates a method for preparing drug eluting balloons using supercritical carbon dioxide impregnation according to an embodiment of the present invention.

FIGS. 3A-3D and 4A-4D show comparisons of balloons before and after treatment by supercritical carbon dioxide impregnation.

### DETAILED DESCRIPTION OF PREFERRED EMBODIMENTS

In preferred embodiments, the balloon material is impregnated with a drug or a compound or mixture containing a drug, preferably by mixing supercritical fluids with the drug or compounds or mixture, to facilitate impregnation, and improve stability and the extraction of the impregnated drug. In a preferred embodiment, the supercritical fluid is supercritical CO₂.

Supercritical carbon dioxide (CO₂) is a special phase of carbon dioxide, that is, when the temperature of carbon dioxide in its pure state exceeds 31 degrees Celsius and the pressure exceeds about 73 atmospheres, carbon dioxide will exist as a supercritical fluid. Carbon dioxide in the supercritical state has the characteristics of both gaseous and liquid substances, and has a large diffusion rate and strong dissolving capacity, which can greatly increase the rate of reaction.

Supercritical CO₂ has many advantages compared to organic solvent methods. Its solvent properties can be significantly adjusted by changing the conditions of pressure and temperature. Various processes based on supercritical fluid technology have received great attention and are increasingly used in the pharmaceutical field as a promising green engineering technology due to the use of less or even no organic solvents, low solvent residues, and many other advantages such as environmental friendliness, mild operating conditions, process stability and controllability.

Thus, using supercritical carbon dioxide technology, embodiments of the present invention provide an uncoated drug eluting balloon, and a method for direct drug loading on the balloon without coating. More specifically, a method according to embodiments of the present invention includes dissolving and uniformly forming (growing) the drug onto the surface of the balloon using supercritical carbon dioxide technology under suitable conditions that maintain the exterior morphology of the balloon, and then releasing the carbon dioxide as a gas by setting suitable depressurization conditions for the carbon dioxide, leaving only the drug on the balloon, thereby forming an uncoated drug eluting balloon with a minimal increase in balloon thickness. Such balloons avoid problems associated with conventional drug-coated balloons such as greater toxicity of vascular cells due to the loss of the coating during delivery in the conventional case, increased drug load, increased thickness of the coating, etc. As a result, the drug eluting balloon according to embodiments of the present invention can pass through blood vessels of smaller diameters and are therefore suitable for small lesion sites.

Embodiments of the present invention provide drug eluting balloons without using conventional coating, where the drug is uniformly loaded on the balloon by using supercritical carbon dioxide technology, which improves the drug loading firmness on the balloon surface, avoids drug loss during vascular delivery, and improves the drug release and absorption effect.

Embodiments of the present invention provides an uncoated drug eluting balloon that avoids solvent residues on the balloon surface caused by the use of other solvents.

Embodiments of the invention provides drug eluting balloons, which are accomplished using a supercritical carbon dioxide impregnation system. The balloon body is formed of a material selected from polyether block amide (PEBA, a block copolymer thermoplastic elastomer comprising rigid polyamide blocks and flexible polyether blocks, known under the tradename PEBAX™), polyvinyl chloride (PVC), polyethylene, polyurethane, nylon, polyethylene terephthalate, etc. The thickness of the balloon is preferably 5 to 15 μm. Methods of making the untreated balloons are generally known in the art and any suitable method may be used. Frequently, the balloon is coated with a hydrophilic polymer layer that serves as a lubricant to facilitate the gliding of the balloon inside the passageways of the body, especially when these are narrowed by some occlusion. These coatings are chosen to be as hydrophilic as possible in order to build a strong interaction with a few layers of water molecules that serve as the true gliding area. Poly-N-Vinyl Pyrrolidone (PNVP or PVP) is frequently used for this purpose, at a thickness ranging from 1 to 5 microns. Often, the said PNVP layer is partially crosslinked, often via a crosslinking agent triggered by UV, so that it is not delaminated from the balloon while travelling inside the passageways. Other polymers like vinylic derivatives of HEMA-PC (2-Methacryloyloxyethyl-2′-(trimethylammoniumethyl)phosphate) can also be used as hydrophilic layers for balloons.

The active drug to be loaded on the balloon may be selected from at least one of sirolimus, sirolimus derivatives, sirolimus analogs, inhibitory RNA, inhibitory DNA, steroids, and complement inhibitors; preferably, the active drug is sirolimus.

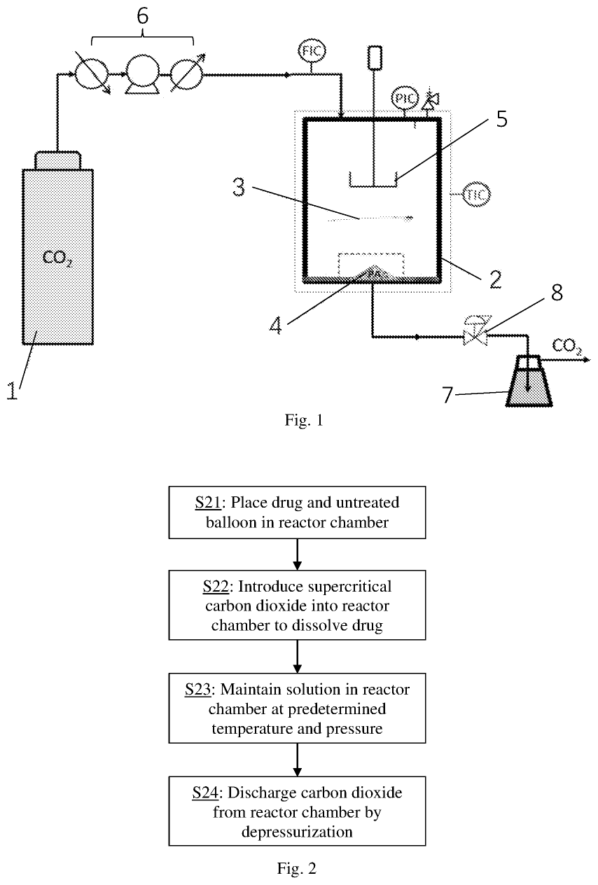

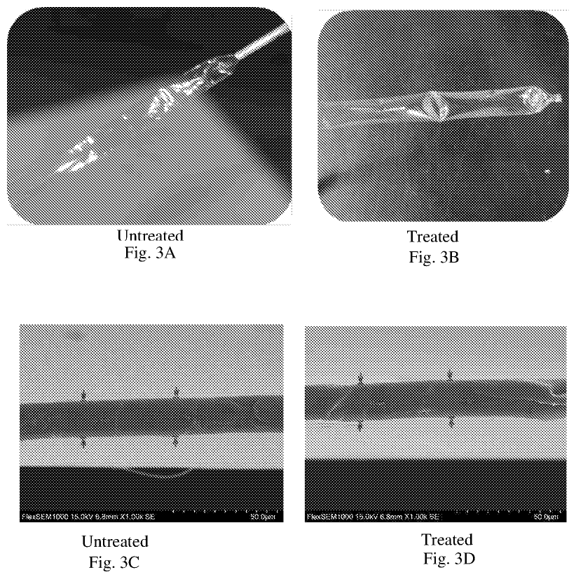

A system and method for making a drug eluting balloon according to an embodiment of the present invention are described with reference to FIG. 1 (system diagram) and FIG. 2 (method flow chart).

As shown in FIG. 1, the system includes a cylinder 1 containing liquid carbon dioxide, and a reactor chamber 2. The drug 4, or a compound or mixture containing the drug, and the balloon 3, are placed in the reactor chamber 2 (step S21). A pump 6 located on the inlet conduit (the conduit between the cylinder 1 and reactor chamber 2) and a pressure regulator 8 on an outlet conduit of the reactor chamber control the pressure of the supercritical carbon dioxide in the reactor chamber 2. A heating element 5 (e.g., a heating agitator) inside or outside of the reactor chamber 2 controls the temperature of the supercritical carbon dioxide. The system further includes a flow indicating control system (FIC), a pressure indicating control system (PIC), and a temperature indicating control system (TIC), respectively configured to monitor the flow on the conduit and the pressure and temperature in the reactor chamber 2.

In operation, the liquid carbon dioxide in the cylinder 1 is transferred into the reactor 2 through the inlet conduit by the pump 6 (step S22). The temperature and pressure in the reactor 2 are maintained so that carbon dioxide exists in a supercritical state. The supercritical carbon dioxide dissolves the drug 4 in the reactor, so that the supercritical carbon dioxide and the drugs form a homogeneous state, and the solution impregnates the balloon 3. The supercritical carbon dioxide is maintained at predefined temperature and pressure for a predetermined length of time to impregnate the balloon (step S23). Then, the carbon dioxide is discharged from the reactor chamber 2 by way of pressure reduction (depressurization) (step S24). More specifically, the supercritical carbon dioxide is depressurized by the pressure regulator 8 to form vapor state, and the active agent is precipitated from the solution in the form of dry powder and collected in a storage bottle 7.

The inventors experimented with various conditions for the supercritical carbon dioxide impregnation process, and determined the preferred conditions for the process. More specifically, balloons made of PEBAX were treated in supercritical carbon dioxide under different conditions, i.e., different combinations of temperatures of 35° C., 45° C. and 55° C., pressures of 100 bar, 175 bar and 250 bar, and durations of 1 h, 12.5 h and 24 h, as listed in the following table.

TABLE I

| | | | Treatment |
|---|---|---|---|
| Experiment | Pressure | Temperature | duration |
| No. | (bar) | (° C.) | (hour) |
| 1 | 100 | 35 | 12.5 |
| 2 | 100 | 45 | 1 |
| 3 | 100 | 45 | 24 |
| 4 | 100 | 55 | 12.5 |
| 5 | 175 | 35 | 1 |
| 6 | 175 | 35 | 24 |
| 7 | 175 | 45 | 12.5 |
| 8 | 175 | 55 | 1 |
| 9 | 175 | 55 | 24 |
| 10 | 250 | 35 | 12.5 |
| 11 | 250 | 45 | 1 |
| 12 | 250 | 45 | 24 |
| 13 | 250 | 55 | 12.5 |
| 14 | 250 | 35 | 24 |

The CO₂ was removed from the reactor 2 at a controlled rate (e.g., 2 bar/min) to avoid foaming of the polymer portion of the balloon.

After treatments were carried out according to the impregnation parameters in the above table, the morphology of the balloon surface was observed by the naked eye, optical microscope, and scanning electron microscope (SEM). The details of two examples are given below.

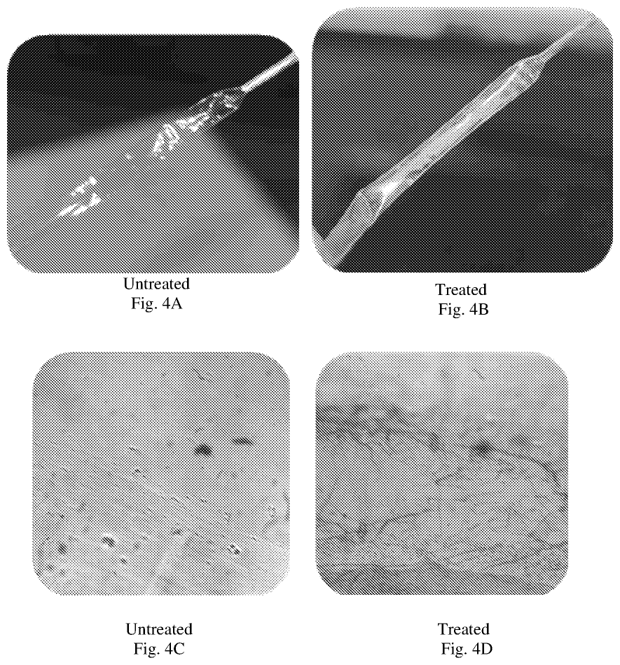

Experiment No. 1: PEBAX balloon, supercritical CO₂ impregnation environment: 35° C., 100 bar, duration 12.5 h. A comparison of the balloon appearance before treatment and after treatment indicates there is no significant change in the surface morphology of the balloons before treatment and after treatment as obtained by visual observation. (FIGS. 3A and 3B show an example of a comparison of untreated vs. treated balloon that have no significant change in surface morphology.) Observation under optical microscope also indicates no significant change. This indicates that the morphology of the balloon body material was not significantly affected under this temperature, pressure and treatment duration, and the drug loading of the balloon under this condition can be further studied.

SEM observation was conducted for the treated and untreated balloons, and it was found that the thickness of the balloon did not change significantly, where the thicknesses of both treated and untreated balloons were about 18 μm. (FIGS. 3C and 3D show an example of a comparison of untreated vs. treated balloon that have no significant change in thickness.)

Experiment No. 14: PEBAX balloon, supercritical CO₂ impregnation environment: 35° C., 250 bar, duration 24 hours. Comparisons of visual observation of balloon appearance before and after treatment (FIGS. 4A and 4B), and of optical microscope observations of balloon appearance before and after treatment (FIGS. 4C and 4D), indicate that under supercritical conditions of 35° C., 250 bar and duration of 24 hours, cracks are evident in the appearance of the treated balloons, indicating balloon material damage.

Through these experiments, it was observed that the surface morphology of the balloons before treatment and after treatment, as obtained by visual and microscopic observation, did not change significantly under the impregnation conditions of experiment Nos. 1˜11. Among these samples, sample No. 2 (45° C., 100 bar, 1 h), 5 (35° C., 175 bar, 1 h) and 8 (55° C., 175 bar, 1 h) had the best surface morphology result under visual and microscopic observations. The conditions in the other experiments (Nos. 12, 13, 14) resulted in significant changes of surface morphology of the balloon, and are therefore not suitable for the balloon treatment method.

Next, the balloon surface drug loading was measured for some of the treated balloon samples, namely, Experiment Nos. 1-14.

The drug loading measurements were carried out using the following procedure.

Instruments: Shimadzu LC-20A High Performance liquid chromatograph; chromatographic column: Sigma Discovery®C18 column: 250×4.6 mm, 5 μm; vortex stirrer; one-hundred-thousandth electronic balance; weighing boat; 0.45 μm needle filter; disposable syringe; sample bottles; ultrasonic instrument; 50 ml volumetric bottle; pipette: 5 ml, 1 ml.

Reagents: methanol (chromatographic pure), acetonitrile (chromatographic pure), water for injection, sirolimus reference.

Solution preparation: For mobile phase preparation:methanol:acetonitrile:water=60:16:24 (v/v/v). 600 mL methanol, 160 mL acetonitrile and 240 mL water were mixed evenly in a suitable solvent bottle. 0.45 m organic filter membrane filtration was applied, and ultrasonic treatment was applied, to obtain the mobile phase. The total volume may be adjusted using the above ratios. For blank solution:acetonitrile.

Chromatographic conditions:

TABLE II

| Velocity | 1.0 | mL/min |
|---|---|---|
| Column temperature | 40° | C. |
| Mobile phase | Methanol - acetonitrile - injection water at | |
| | 60:16:24 (v/v/v) | |
| Sample volume | 20 | μL |
| Elapsed time | 30 | min |
| Detection wavelength | 280 | nm |

| | |
|---|---|
| Washing solvent | acetonitrile |

Reference solution preparation: 25 mg sirolimus reference was accurately weighed and placed in a 50 mL volumetric flask, and acetonitrile was added to about 2/3 volume, and shaken until completely dissolved. The solution was diluted with acetonitrile to the scale, shaken well, to obtain the reference reserve solution (target concentration 500 μg/mL). 1.0 ml intermediate reference solution was accurately transferred to a 50 mL measuring bottle, and a diluent was added to the scale to dilute it, and shaken well, to obtain the working reference solution (target concentration 10 μg/mL). (Note: The reference solution is stable within 7 days at 5 T (refrigerator condition 2-8 T) or room temperature.)

Sample solution preparation: 2 mL water was added to the balloon sample, mixed with a vortex stirrer for 3 mins, then 3 mL acetonitrile was added. 1 ml balloon sample solution was then taken, and 3 mL acetonitrile was added to it, then ultrasonic treatment was applied for 15 min. The sample solution was then filtered by 0.45 μm membrane before injection.

Analysis procedure: The system was balanced until a flat baseline was obtained. (Note: If necessary, a blank can be injected before waiting for a sample.)

Calculation: The concentration X of the drug in the balloon sample solution was calculated using:

$$Concentration X = \frac{{A}_{T} \times {C}_{S} \times V}{{A}_{S}}$$

where A_{T} is the area under the sirolimus peak for the balloon sample solution, Aₛ is the average area under the peak for the sirolimus reference solution, C_{S} is the reference solution concentration (μg/mL), and V is the dilution volume (mL). (Note: The sirolimus peak area refers to the sum of the peak areas of sirolimus and its isomers in the chromatogram.)

As mentioned earlier, it was determined that among all the tested conditions, the best treatment condition, based on the microscopic observations, was No. 2 (45° C., 100 bar, 1 h), 5 (35° C., 175 bar, 1 h) and 8 (55° C., 175 bar, 1 h). However, among the experiments where the surface morphology of the balloon was satisfactory, medium pressure, higher temperature and longer treatment time, such as 175 bar, 45/55° C. and 12.5/24 hours (Experiment No. 7 and 9), achieved higher drug loading.

### Drug Loading

The measured drug loading for the three balloon samples are summarized below:

TABLE III

| | Mass sirolimus | Drug loading |
|---|---|---|
| Treatment condition | (μg) | (μg/mm²) |
| (No. 1) 35° C., 100 bar, 12.5 h | 58.96 | 0.43 |
| (No. 2) 45° C., 100 bar, 1 h | 24.67 | 0.18 |
| (No. 3) 45° C., 100 bar, 24 h | 46.07 | 0.33 |
| (No. 4) 55° C., 100 bar, 12.5 h | 17.98 | 0.13 |
| (No. 5) 35° C., 175 bar, 1 h | 13.74 | 0.10 |
| (No. 6) 35° C., 175 bar, 24 h | 9.26 | 0.07 |
| (No. 7) 45° C., 175 bar, 12.5 h | 180.94 | 1.31 |
| (No. 8) 55° C., 175 bar, 1 h | 45.86 | 0.33 |
| (No. 9) 55° C., 175 bar, 24 h | 196.17 | 1.42 |
| (No. 10) 35° C., 250 bar, 12.5 h | 100.42 | 0.73 |
| (No. 11) 45° C., 250 bar, 1 h | 10.75 | 0.08 |
| (No. 12) 45° C., 250 bar, 24 h | 38.69 | 0.28 |
| (No. 13) 55° C., 250 bar, 12.5 h | 117.46 | 0.85 |
| (No. 14) 35° C., 250 bar, 24 h | 104.26 | 0.76 |

Drug load of currently commercially available balloons are between about 0.8-2.0 μg/mm². Thus, balloon the preparation method described above using supercritical carbon dioxide technology can be used to achieve the drug load of commercially available balloon, while reducing the balloon diameter.

Next, the ex vivo drug release was measured for one treated balloon samples, namely, Experiment Nos. 7. The balloon is assembled with delivery system, and the balloon is folded on the folding machine.

The drug loading measurements were carried out using the following procedure.

Typically, a freshly explanted swine artery segment (˜3 cm) was dissected and flushed with normal saline. Then the balloon was inflated to attach to artery wall well, held for 60s and then deflated. Following removal of the balloon, the segment of the artery was cut to 4 parts and flushed with PBS for 15 min/1 h/2 h/4 h at a rate of 4 mL/min to wash away the desorbed drug. Then balloon was cut for remained drug dose testing. The artery tissue was ground, drug was extracted with acetonitrile and tested by HPLC.

Instruments: Jing Xin JXFSTPRP-L Grinder; Shu Ke TGL-1850 Centrifuge

Treatment of blood vessels: Place the treated blood vessel in a 5 ml thickened centrifuge tube, add a certain amount of acetonitrile (1 ml at present), put it into a grinder for grinding (60 Hz grinding for 4 min), completely grind it and centrifuge it at 10000 rpm for 3 min, take the supernatant and filter it through a 0.45 um membrane, and take the continuous filtrate for test in HPLC (the same protocol as drug loading on balloon).

Treatment of used balloon: the same treatment as drug loading on balloon.

Ex vivo drug release result:

TABLE IV

| Artery sample | Drug concentration in tissue (ng/g) |
|---|---|
| SC-CO2-flushing-15 min. | 119117.62 |
| SC-CO2-flushing-1 h. | 37912.87 |
| SC-CO2-flushing-2 h. | 34747.75 |
| SC-CO2-flushing-4 h. | 25710.03 |
| Balloon sample | Remained drug dose on balloon (μg) |
| Acetonitrile-HPLC | 34.19 |

After the balloon adhered to the wall, there was a large tissue drug concentration within 15 minutes, indicating that the drug could be transferred to the blood vessel wall. With the prolongation of washing time, the dosage decreased significantly, but remained above 10000 ng/g (effective drug concentration).

In summary, loading of certain active drugs on balloons can be achieved using supercritical carbon dioxide technology and drug with effective concentration could transfer and penetrate in artery wall during balloon expansion in artery, and thus uncoated drug balloons can be prepared using this technology.

It will be apparent to those skilled in the art that various modification and variations can be made in the drug eluting balloon preparation method and related apparatus of the present invention without departing from the spirit or scope of the invention. Thus, it is intended that the present invention cover modifications and variations that come within the scope of the appended claims and their equivalents.

What is claimed is:

1. A drug eluting balloon loaded with a drug without added coating to host the drug.

2. The drug eluting balloon of claim 1, wherein the drug is sirolimus.

3. The drug eluting balloon of claim 1, wherein a drug loading of the balloon is in a range 0.8 to 3.0 micrograms per mm².

4. A method of preparing a drug eluting balloon, comprising:
preparing a balloon;
dissolving a drug or a compound or mixture containing the drug in a supercritical carbon dioxide;
impregnating the balloon with the supercritical carbon dioxide having the drug dissolved therein in a reactor chamber; and
removing the supercritical carbon dioxide from the reactor chamber by depressurization.

5. The method of claim 4, wherein the balloon is formed of a polyether block amide.

6. The method of claim 4, wherein the balloon is coated with a hydrophilic layer, at a thickness between 1 and 5 microns.

7. The method of claim 6, wherein the hydrophilic layer is polyN-vinylpyrrolidone.

8. The method of claim 4, wherein the balloon is formed of a material selected from polyether block amide, polyvinyl chloride (PVC), polyethylene, polyurethane, nylon, and polyethylene terephthalate.

9. The method of claim 4, wherein in the impregnating step, the supercritical carbon dioxide is maintained at a temperature between 35 and 55° C., and a pressure between 100 and 250 bars.

10. The method of claim 9, wherein the impregnating step is maintained for a time duration of 1 hour to 24 hours.

11. The method of claim 4, wherein the drug is selected from of sirolimus, sirolimus derivatives, sirolimus analogs, inhibitory RNA, inhibitory DNA, steroids, and complement inhibitors.

12. The method of claim 4, wherein the drug is sirolimus.
